(19) United States
(12) Patent Application Publication
(10) Pub. No.: US 2003/0134340 A1
(43) Pub. Date: Jul. 17, 2003

(54) Method for detecting pathogenic prion proteins by means of mass spectroscopy

(76) Inventor: Thomas Lengsfeld, Marburg (DE)

Correspondence Address: Finnegan, Henderson, Farabow,, Garrett & Dunner, L.L.P., 1300 I Street, N.W., Washington, DC, 20005-3315, US

(21) Appl. No.: 10/345,148

(22) Filed: Jan. 16, 2003

(30) Foreign Application Priority Data
Jan. 17, 2002 (DE) 102 01 777.8

(51) Int. Cl. G01N 33/53; G01N 33/542

(52) U.S. Cl. 435/7.9

(57) ABSTRACT

A method for detecting one or more pathogenic prion proteins in a sample, which can be of a body fluid of human or animal origin, and which contains a PrP protein that assumes a natural, nonpathogenic conformation, PrP^{C}, and a pathogenic conformation, termed PrP^{Sc}, is described. The method can comprise: providing a sample suspected of containing the pathogenic form of at least one prion protein; exposing the sample to a chemical agent under conditions where the chemical agent and the prion protein or proteins react to form at least one covalent bond involving the prion protein or proteins; and mass-spectroscopically analyzing the resulting prion protein or proteins to detect the presence of the pathogenic form of the prion protein or proteins; wherein at least one additional peak is observed in the mass spectrum when the pathogenic form of a prion protein is present.

The invention relates to a method for detecting one or more pathogenic (i.e., pathological) prion proteins in a sample, such as that of a body fluid of human or animal origin, by means of a mass-spectroscopic method.

Prion diseases, such as Creutzfeldt-Jakob disease (CJD), can develop as a result of inherited genetic defects, or can be acquired by way of routes of infection that are not yet completely understood. In addition, they occur as spontaneous, or “sporadic”, forms, which are postulated to be due to a somatic mutation in the gene for the prion protein (Prusiner, Proc. Natl. Acad. Sci. U.S.A., 95:13363-13383 (1998)). Iatrogenic routes of infection result, for example, from treatment with prion-contaminated growth hormones, sex hormones, or corneal and meningeal transplants. The use of inadequately sterilized surgical material also represents a possible source of infection.

The prion proteins (abbreviated to PrP), which are from 33 to 35 kD in size, are found in a natural physiological isoform (PrP^{C}) and in a pathologically infectious isoform (PrP^{Sc}), with the infectious isoform arising from the noninfectious physiological form as the result of a refolding of the secondary and tertiary structures. PrP^{Sc} is very probably the only material component of the prions which is required for the transmission and pathogenesis of the prion diseases (Prusiner, Proc. Natl. Acad. Sci. U.S.A., 95:13363-13383 (1998)).

It is already known from Prusiner et al., Cell 38:127 (1984) and Biochemistry 21:6942 (1982) that prion proteins are accessible to partial proteolysis. Since then, it has been found that PrP^{C} is virtually completely accessible to proteolysis, whereas PrP^{Sc} can only be degraded down to a size of from 27 to 30 kD.

The protein form that is not accessible to further proteolysis is termed a protease-resistant core, and is designated PrP²⁷⁻³⁰. It is formed as a result-of the detachment of approximately 67 amino acids from the NH₂ terminus, and is itself composed of approximately 141 amino acids.

Some methods for detecting the pathological prion isoforms are already known. Barry and Prusiner, J. Infect. Dis. 154:518-521 (1986), for example, describe a Western blot test using a monoclonal anti-prion protein antibody (Mab) 13A5. This hamster PrP-specific Mab was isolated in mice that had been immunized with purified, denatured PrP²⁷⁻³⁰, which had been isolated from scrapie-infected hamsters.

Other antibodies, which, like Mab 13A5, are directed both against PrP^{C} and against PrP^{Sc} (provided this latter is present in denatured form) are disclosed in U.S. Pat. No. 4,806,627. Furthermore, immunizations have been carried out using recombinant prion proteins that have been expressed in bacteria, as described, for example, in Zanusso et al., Proc. Natl. Acad. Sci. USA, 95:8812-8816 (1998). It has likewise been possible to prepare monoclonal antibodies by means of peptide immunization, as described, for example, in Harmeyer et al., J. Gen. Virology, 79:937-945 (1998), and by means of nucleic acid immunization, as explained in Krasemann et al., J. Biotechnology, 73:119-129 (1999).

U.S. Pat. No. 4,806,627 mentioned another application of these antibodies apart from Western blotting, namely what is termed an ELISA (enzyme-linked immunosorbent assay). In this ELISA, prions that had been fixed on a microtiter plate were bound by the Mab 3F4, and this antibody was then detected by means of a second antibody, which catalyzes a color reaction by way of an enzyme which is coupled to it.

In all these detection methods, the sample is pretreated with the enzyme proteinase K in order to remove normal prion protein that is present in the sample. Proteinase K is also added to ensure that only the protease-resistant, pathogenic prion protein is detected, since the antibodies can, of course, also bind the normal prion protein with a high degree of affinity.

International patent application WO 98/37411 discloses a detection method that can be used to detect the pathogenic conformation of prion proteins in a sample. In this method, the sample is divided into two portions. The first portion is bound to a solid support and then contacted with a labeled antibody. This antibody binds to the nonpathogenic form of prion proteins with a higher affinity than it does to the nondenatured, pathogenic form of the proteins. The second portion of the sample is then subjected to a treatment that alters the conformation of the pathogenic prion proteins, resulting in the accessibility, and consequently the affinity for the labeled antibody, being drastically increased. The second sample, which has been treated in this way, is then brought into contact with a second support and reacted with a labeled antibody. The quantities of the labeled antibody that are bound in the first portion and in the second portion are then measured and compared with each other. The difference between the two measurement results indicates whether the pathogenic form of the prion proteins was present in the sample. This detection method is termed a conformation-dependent immunoassay, and is abbreviated CDI. The sensitivity of the CDI can be increased if the sample is subjected to a pretreatment with a proteolytic enzyme, for example proteinase K or dispase. The treatment with proteases destroys PrP^{C} and nonrelevant proteins in the sample, and the protease-resistant PrP²⁷⁻³⁰ is left in the sample.

Examination of human blood plasma for the presence of the pathogenic prion proteins requires very sensitive and specific detection systems, which should be suitable for automation. The detection is made more difficult by the fact that the physiological bases for the pathological effect of prions are still not known.

German patent application 101 52 677.6 has recently described, for the first time, antibodies for specifically detecting pathogenic prions of human origin. This detection method uses monoclonal antibodies from the hybridoma cell lines DSM ACC 2522, DSM ACC 2523, and DSM ACC 2524, which are able, in a conformation-dependent immunoassay method, to distinguish the nonpathological conformation of human prion proteins from the pathological conformation of human prion proteins.

Despite all the methods for detecting pathogenic proteins that have thus far been developed, there is still a substantial need to have available additional, rapidly implementable, reliable, and highly sensitive methods for detecting pathogenic prions.

Surprisingly, it has been found that reacting a mixture comprising one or more prion proteins having pathological and nonpathological conformations with a chemical agent that is suitable for producing additional covalent bonds in the prion proteins gives rise, in a conformation-dependent manner, to molecules that generate signals that can be distinguished mass-spectroscopically.

A method that is based on this finding and whose purpose is to detect one or more pathogenic prion proteins in a sample, such as a sample of a body fluid that is of human or animal origin and contains at least one PrP protein which is able to assume a natural nonpathological conformation, PrP^{C}, and a pathological conformation, PrP^{Sc}, can be carried out by,

providing a sample suspected of containing at least one prion protein, which can also be chemically modified;

reacting the prion protein or proteins with a chemical agent, wherein the reacting results in the formation of covalent bonds; and

mass-spectroscopically analyzing the prion protein or proteins which are thereby chemically modified, wherein at least one additional peak is observed in the mass spectrum when one or more pathogenic prion proteins are present in the sample.

For example, in an embodiment, the method is a method for detecting a pathogenic form of at least one prion protein in a sample suspected of containing one or more prion proteins that assume a natural, nonpathogenic conformation, and a pathogenic conformation. The method comprises

providing a sample suspected of containing the pathogenic form of at least one prion protein;

exposing the sample to a chemical agent under conditions where the chemical agent and the prion protein or proteins react to form at least one covalent bond involving the prion protein or proteins; and

mass-spectroscopically analyzing the resulting prion protein or proteins to detect the presence of the pathogenic form of the prion protein or proteins;

wherein at least one additional peak is observed in the mass spectrum when the pathogenic form of one or more prion proteins is present.

The method of the invention can detect a single prion protein in a sample as well as multiple copies of the same prion protein. Likewise, it can detect two or more different prion proteins, each of which can be independently present in the sample in amounts of one or more copies.

In order to increase the sensitivity and reduce or eliminate possible interferences, it is possible

to contact the sample with a support substance that adsorbs one or more prion proteins;

separate the adsorbed prion protein or proteins from the remainder of the sample;

react the prion protein or proteins, in the adsorbed state or following release, with a chemical agent, with the formation of covalent bonds, and

mass-spectroscopically analyze the prion protein or proteins, which are thereby chemically modified.

For example, in one embodiment, the method for detecting a pathogenic form of at least one prion protein discussed above can further comprise contacting the sample suspected of containing the pathogenic form of at least one prion protein with a support substance that adsorbs one or more prion proteins. This contacting can be performed before reacting the prion protein or proteins with the chemical agent, and can result in binding of the prion protein or proteins to the support substance.

In other embodiments of the invention, the method can further comprise separating the adsorbed prion protein or proteins from the remainder of the sample. Likewise, it can further comprise releasing the adsorbed prion protein or proteins from the support substance. Either the bound or released protein or proteins can be used for detection by mass-spectroscopy.

In the method of the invention, when at least one pathogenic prion is present, at least one further peak is observed in the mass spectrum when compared with the nonpathogenic form of the prion protein.

In embodiments, the sample suspected of containing one or more prion proteins is a body fluid. The sample can be provided as the body fluid itself, or in another form that includes other components. For example, it can be provided as a diluted sample, diluted with any suitable diluent, such as water, saline, or phosphate-buffered saline. Likewise, it can be mixed with one or more buffers or reaction mixtures suitable for antibody binding reactions, adsorption to solid supports, etc. The choice of diluents and other components can be made by those practicing the invention, based on the desired reaction to be achieved. Furthermore, the sample, and in particular the prion proteins present in the sample, can be chemically modified prior to exposure to the chemical agent that will chemically modify the prion proteins. For example, the sample, and in particular the proteins in the sample, can be covalently modified before being reacted with the chemical agent. Suitable reagents for chemically modifying the prion proteins before reaction with the chemical agent include, but are not limited to, proteinase K and dispase.

The body fluid can be any body fluid, including, but not limited to, body fluids of human or animal origin. The body fluid can be, for example blood, serum, plasma, urine or milk. Likewise, it can be fluidized organs or organ tissues, such as brain tissue, lymph nodes, tonsils, or muscles.

Any suitable support substance can be used. For example, agarose, a chromatography resin, a microtiter plate, or a nitrocellulose or polyamide membrane can be employed as the support substance for the adsorption. The support substance can be coated with an agent for binding prions. Suitable agents of this nature are lysozyme or one of its fragments, a prion-binding monoclonal or polyclonal antibody or one of its fragments, or another compound which possesses prion-binding ligands.

The support can be contacted with the sample, such as a body fluid, that is to be investigated for the presence of one or more pathogenic prions. The prion or prions that are fixed on the support can then be used for the detection method according to the invention, either directly or after the prion or prions have been eluted from the support.

The detection method according to the invention is based, at least in part, on the insight that, while having the same molecular composition, natural, nonpathological prions differ from the pathological conformation of the prions, i.e. PrP^{Sc}, in their spatial structure. For this reason, they present qualitatively and quantitatively different functional groups on their surface for a reaction with a chemical agent. If, therefore, a mixture of pathological and nonpathological prions is brought into contact with a chemical agent, for example, with an oxidizing or reducing agent, or with an alkylating or acylating agent, the chemical agent will come across functional groups on the prion surface that are qualitatively and quantitatively different, and will therefore enter into a different number of bonds with the nonpathological prion on the one hand, and with the pathological prion on the other hand. As a consequence of this, the masses of the reaction products, obtained with a particular chemical agent, of nonpathological prions differ enough from those of patholotical prions that the two can be distinguished mass-spectroscopically.

If the sample that is to be investigated only contains one or more nonpathological prions, it is then only possible to detect one integrated peak, or a group of closely related peaks, in the mass spectrum. However, if the sample being investigated also contains one or more pathogenic prions, a divergent mass spectrogram is obtained. In addition to the peak that is characteristic for the nonpathological prion or prions, there then appears at least one further peak, or a group of further peaks, which is characteristic for the reaction of the chemical agent employed with the pathological prion or prions.

Any substance that is able to react with the functional groups appearing on the surface of a prion or of prions is suitable for use as a chemical agent. The substance can be an oxidizing agent or reducing agent. Non-limiting examples of suitable oxidizing agents are H₂O₂, Cu⁺⁺/ascorbate, and Fe⁺⁺⁺/ascorbate. A non-limiting example of a suitable reducing agent is NaBH₄.

Differences in the masses of the reaction products obtained with one or more nonpathogenic prions and one or more pathogenic prions can also be achieved by reacting with alkylating agents or acylating agents. A non-limiting example of a suitable alkylating agent is formaldehyde. A non-limiting example of a suitable acylating agent is a dicarboxylic anhydride, such as succinic anhydride.

It has been found that prions also exhibit special side chains on their surfaces. These side chains are characterized by cysteine or methionine residues, by aspartic acid or glutamic acid residues, or by asparagine or glutamine residues, and also lysine or arginine residues.

Non-limiting examples of agents that are suitable for reacting with side chains which are modified in this way are maleic anhydride (for modifying the SH-cysteine residues), diazoacetamide (for reacting with glutamic acid, aspartic acid esters, and cysteine residues), and 1,2-cyclohexanedione (for reacting with arginine residues). Other suitable agents for modifying amino acids, and particularly their side chains, are known to those of skill in the art, and can be used according to the invention without undue experimentation.

The reliability and sensitivity of the detection method according to the invention were demonstrated by adding quite small quantities of nonpathogenic and pathogenic prions to groups of 10 and 100 plasma samples. In all cases, it was possible to reliably detect the pathogenic prions alongside the nonpathogenic prions in a mass spectrogram. Indeed, the method can detect a single prion protein, in either the nonpathogenic or pathogenic form, in a sample.

The implementation of the detection method according to the invention is illustrated by the following examples that show detection of one or more prion proteins following oxidation.

### EXAMPLE 1

Prion proteins of differing conformation and differing origin were added to plasma protein solutions. The prion proteins were diluted down to nanomolar to femtomolar concentrations. The prion proteins were immunoprecipitated with a mixture of prion-specific antibodies. The immunoprecipitated proteins were dissolved (10 mg of protein/ml in oxidation buffer (50 mM Hepes buffer, pH 7.4; 100 mM KCl; 10 mM MgCl₂)) and then treated by means of metal-catalyzed oxidation (MCO). For this, 25 mM ascorbic acid and 100 μM FeCl₃ were added to 750 μl of protein solution. The reaction mixture was incubated at 37° C. for 12 h and the oxidation reaction was then stopped by adding EDTA solution. The prion proteins were then characterized mass-spectrometrically. This resulted in a prion type-specific chromatogram.

### EXAMPLE 2

Prion proteins of differing conformation and differing origin were added to plasma protein solutions (10 mg of protein/ml in oxidation buffer (50 mM Hepes buffer, pH 7.4; 100 mM KCl; 10 MM MgCl₂)). The prion proteins were diluted down to nanomolar to femtomolar concentrations. The protein solution was subsequently treated by means of metal-catalyzed oxidation (MCO). For this, 25 mM ascorbic acid and 100 μM FeCl₃ were added to 750 μl of protein solution. The reaction mixture was incubated at 37° C. for 12 h and the oxidation reaction was then stopped by adding EDTA solution. The prion proteins were immunoprecipitated with a mixture of prion-specific antibodies and then characterized mass-spectrometrically. This resulted in a prion type-specific chromatogram.

### EXAMPLE 3

Prion proteins of differing conformation and differing origin were added to plasma protein solutions. The prion proteins were diluted down to nanomolar to femtomolar concentrations. The prion proteins were bound to a support using a mixture of prion-specific antibodies. The bound proteins were treated on the support with oxidation buffer (50 mM Hepes buffer, pH 7.4; 100 mM KCl; 10 mM MgCl₂) and metal-catalyzed oxidation (MCO). For this, 25 mM ascorbic acid and 100 μM FeCl₃ were added to the support. The reaction mixture was incubated at 37° C. for 12 h and the oxidation reaction was then stopped by adding EDTA solution. The bound and oxidized prions were then characterized mass-spectrometrically. This resulted in a prion type-specific chromatogram.

### EXAMPLE 4

Prion proteins of differing conformation and differing origin were added to plasma protein solutions. The prion proteins were diluted down to nanomolar to femtomolar concentrations. The prion proteins were immunoprecipitated with a mixture of prion-specific antibodies. The immunoprecipitated proteins were dissolved (10 mg of protein/ml in oxidation buffer (50 mM Hepes buffer, pH 7.4; 100 mM KCl; 10 mM MgCl₂)) and then treated by means of metal-catalyzed oxidation (MCO). For this, 25 mM ascorbic acid and 100 μM FeCl₃ were added to 750 μl of protein solution. The reaction mixture was incubated at 37° C. for 12 h and the oxidation reaction was then stopped by adding EDTA solution. The oxidized proteins were derivatized with 2,4-dinitrophenylhydrazine. The prion proteins were then characterized mass-spectrometrically. This resulted in a prion type-specific chromatogram.

What is claimed is:

1. A method for detecting a pathogenic form of at least one prion protein in a sample suspected of containing one or more prion proteins that assume a natural, nonpathogenic conformation, and a pathogenic conformation, said method comprising
providing a sample suspected of containing the pathogenic form of at least one prion protein;
exposing the sample to a chemical agent under conditions where the chemical agent and the prion protein or proteins react to form at least one covalent bond involving the prion protein or proteins; and
mass-spectroscopically analyzing the resulting prion protein or proteins to detect the presence of the pathogenic form of the prion protein or proteins;
wherein at least one additional peak is observed in the mass spectrum when the pathogenic form of one or more prion proteins are present.

2. The method of claim 1, wherein the sample is a sample of a body fluid.

3. The method of claim 2, wherein the body fluid is of human or animal origin.

4. The method of claim 2, wherein the body fluid is blood, serum, plasma, urine, or milk.

5. The method of claim 2, wherein the body fluid is a fluidized organ or organ tissue.

6. The method of claim 5, wherein the organ or organ tissue is brain, lymph nodes, tonsils, or muscles.

7. The method of claim 1, wherein the prion protein or proteins present in the sample are chemically modified before being reacted with the chemical agent.

8. The method of claim 7, wherein the proteins are chemically modified by treating them with proteinase K or dispase.

9. The method of claim 1, wherein the method detects a single prion protein.

10. The method of claim 1, wherein the method detects multiple different prion proteins.

11. The method of claim 1, further comprising contacting the sample with a support substance that adsorbs one or more prion proteins before reacting the prion protein or proteins with the chemical agent, resulting in binding of the prion protein or proteins to the support substance.

12. The method of claim 11, further comprising separating the adsorbed prion protein or proteins from the remainder of the sample.

13. The method of claim 12, further comprising releasing the adsorbed prion protein or proteins from the support substance.

14. The method of claim 11, wherein the support substance is agarose, a chromatography resin, a microtiter plate, or a nitrocellulose or polyamide membrane.

15. The method of claim 11, wherein the support substance is coated with lysozyme or one of its fragments, with a polyclonal or monoclonal antibody or one of its fragments, or with another compound that possesses prion protein-binding ligands.

16. The method of claim 11, wherein the prion protein or proteins that are adsorbed on the support substance are used for detecting the pathogenic form of the prion protein or proteins.

17. The method of claim 11, wherein the prion protein or proteins are eluted from the support substance, and the eluted prion protein or proteins are used for detecting the pathogenic form of the prion protein or proteins.

18. The method of claim 1, wherein the chemical agent is an oxidizing agent or a reducing agent.

19. The method of claim 1, wherein the chemical agent is an alkylating agent.

20. The method of claim 1, wherein the chemical agent is an acylating agent.

21. The method of claim 1, wherein the chemical agent specifically covalently modifies one or more cysteine residues, one or more methionine residues, or both.

22. The method of claim 1, wherein the chemical agent specifically modifies one or more aspartic acid residues, one or more glutamic acid residues, or both.

23. The method of claim 1, wherein the chemical agent specifically modifies one or more asparagine residues, one or more glutamine residues, or both.

24. The method of claim 1, wherein the chemical agent specifically modifies one or more lysine residues, one or more arginine residues, or both.
